(19) United States
(12) Patent Application Publication
(10) Pub. No.: US 2003/0147810 A1
(43) Pub. Date: Aug. 7, 2003

(54) Compositions and methods for reporting of protease activity within the secretory pathway

(75) Inventors: Brian D. Ross, Ann Arbor, MI (US); Alnawaz Rehemtulla, Plymouth, MI (US)

Correspondence Address: GREGORY P. EINHORN, Fish & Richardson P.C., Suite 500, 4350 La Jolla Village Drive, San Diego, CA, 92122, US

(73) Assignee: University of Michigan, Ann Arbor, MI (US)

(21) Appl. No.: 10/066,319

(22) Filed: Feb. 1, 2002

(51) Int. Cl. C12Q 1/68; A01K 67/00; C07H 21/04; C12N 9/10; C12N 9/64; C12N 1/18; C12P 21/02; C12N 5/06; C12N 5/04

(52) U.S. Cl. 424/9.6; 435/6; 435/69.1; 435/226; 435/320.1; 435/317.1; 435/325; 435/252.3; 536/23.2; 435/254.2; 435/419; 800/8

(57) ABSTRACT

The present invention provides chimeric polypeptides and nucleic acids encoding the chimeric polypeptides and methods for using the same to detect and measure protease activity. It further provides in vivo and in vitro methods for identifying modulators of protease activity, e.g., by high throughput assays.

### TECHNICAL FIELD

This invention generally pertains to the fields of medicine and drug screening. In one aspect, the invention provides a high-throughput screening assay to identify compounds for the prevention or amelioration of Alzheimer's disease.

### BACKGROUND

Alzheimer's disease (AD) is a progressive, degenerative disease that attacks the brain. Alzheimer's disease is a neurodegenerative disorder characterized by accumulation of amyloid plaques and neurofibrillary tangles in the brain. It results in impaired memory, thinking and behavior. It affects an estimated 4 million American adults. Annually, more than 100,000 Americans die as a result of AD, making it the fourth leading cause of death in adults after heart disease, cancer and stroke.

Alzheimer's is an illness that develops gradually. Early symptoms include difficulty remembering recent events and performing familiar tasks. The afflicted may encounter confusion, personality changes, altered behavior and judgment. They may have trouble finding words, finishing thoughts or following directions. How quickly the illness progresses differs from person to person. Unfortunately, the disease eventually leaves its victims completely unable to care for themselves. This disease is a tremendous burden on the health care system and society in general.

Biochemically, Alzheimer's disease is characterized by the progressive formation in the brain of insoluble amyloid plaques and vascular deposits comprising the 4 kilodalton (kD) amyloid β-peptide (Aβ). Amyloid P-peptide overproduction has been suggested as being the cause of familial early-onset Alzheimer's disease. Formation of amyloid β-peptide requires proteolytic cleavage of a large type-1 transmembrane protein, the β-amyloid precursor protein (APP), which is constitutively expressed in many cell types. To initiate amyloid β-peptide formation, β-secretase cleaves APP at the NH2-terminus to release a 100 kD soluble fragment and a 12 kD COOH-terminal fragment, C99, that remains membrane bound. Subsequent cleavage of the cell associated COOH-terminal fragment by γ-secretase results in the formation of amyloid β-peptide.

Vassar (1999) Science 286:735-741, described the cloning and expression of a protease termed BACE (for beta-site APP-cleaving enzyme). Overexpression of the protease increased the amount of beta-secretase cleavage products. These products were cleaved exactly and only at known beta-secretase positions. Antisense inhibition of endogenous BACE messenger RNA decreased the amount of beta-secretase cleavage products. Purified BACE protein cleaved APP-derived substrates with the same sequence specificity as beta-secretase.

Roberds (2001) Hum. Mol. Genet. 10:1317-1324, described the generation of two lines of BACE knockout mice. They characterized these mice for pathology, beta-secretase activity and amyloid β-peptide production. The mice appeared to develop normally and showed no consistent phenotypic differences from their wild-type littermates. They had overall normal tissue morphology and brain histochemistry, normal blood and urine chemistries, normal blood-cell composition, and no overt behavioral and neuromuscular effects. Brain and primary cortical cultures from BACE knockout mice, however, showed no detectable β-secretase activity. Primary cortical cultures from BACE knockout mice produced much less amyloid β-peptide from APP. The findings that BACE is the primary β-secretase activity in the brain and that loss of β-secretase activity produces no profound phenotypic defects with a concomitant reduction in β-amyloid peptide clearly indicate that BACE is an excellent therapeutic target for the treatment of AD.

The discovery that BACE is a β-secretase associated with AD has motivated the need to develop specific inhibitors of this enzyme. Unlike the better known aspartic proteinases, cathepsin D and HIV-1 proteinase, both of which are either cytosolic or extracellular, BACE is unique in that it is a Golgi retained enzyme. The Golgi represents a specific intracellular compartment and a constituent of the secretory pathway. The fact that BACE is a Golgi resident proteinase brings into play a number of specific issues with regards to screening for inhibitors and to monitoring BACE activity in tissue culture cells and in animal models. Firstly, although a large amount of structural and functional (inhibitor studies) data is available for many aspartic proteinases, development of inhibitors for BACE remains a challenge because the lead compound will have to survive the perils of the peripheral circulation, cross the blood-brain barrier, permeate into neurons and reach the Golgi compartment. These challenges necessitate the ability to monitor BACE activity non-invasively in tissue culture cells and in animal models. Secondly, when measuring BACE activity in cells, one cannot simply add synthetic peptidyl substrates to cells and expect them to diffuse into the Golgi compartment where BACE residues. For similar reasons, measurement of BACE activity in animal models would also pose a major challenge. The ability to monitor BACE activity non-invasively in animal models, however, would greatly facilitate testing the efficacy of lead compounds as BACE inhibitors. In addition, the ability to monitor BACE activity in tissue culture cells (as opposed to using a purified enzyme) would provide an ideal high-throughput screening assay for BACE inhibitors, since factors such as cell permeability and ability of the candidate inhibitor to enter the Golgi compartment would be taken into consideration as part of the screening assay.

### SUMMARY

In one aspect, the invention provides chimeric polypeptides and chimeric nucleic acids encoding the polypeptides, wherein the polypeptides include a first, a second and a third domain. The first domain includes a Golgi retention signal peptide or an endoplasmic reticulum (ER) retention signal peptide. The second domain includes a protease cleavage site. In some embodiments of the invention, the second domain may possess a sequence encoding two or more protease cleavage sites. The third domain includes a reporter molecule. The protease cleavage site is between the Golgi retention signal peptide and the reporter molecule.

The first domain coding sequence can be upstream of the second domain coding sequence and the third domain coding sequence. Alternatively, the third domain coding sequence can be upstream of the second domain coding sequence and the first domain coding sequence. The nucleic acid encoding the chimeric polypeptide can include a promoter. The promoter can be operably linked to the nucleic acid. The promoter can be a constitutive promoter or an inducible promoter.

Embodiments of the invention can include and endoplasmic reticulum (ER) or Golgi retention signal peptides that can be mammalian, yeast or viral. An example of a mammalian ER or Golgi retention signal peptide is a human ER or Golgi retention signal peptide. ER retention signal peptides can contain sequence motifs such as KDEL (SEQ ID NO: 1) and NEFA (SEQ ID NO:2).

In some embodiments, the Golgi retention signal peptide can be a Golgi retention signal peptide from a Golgi resident enzyme. For example, the Golgi resident enzyme can be Golgi glycosyltransferase. The Golgi glycosyltransferase can be a glucosaminyltransferase I (GlcNAcTI), a beta 1,4-galactosyltransferase (GaIT) or an alpha 2,6-sialytransferase (ST).

Embodiments of the invention can include a protease cleavage site that is a secretase cleavage site. The secretase cleavage site can be a beta-secretase cleavage site and/or a gamma-secretase cleavage site. Examples of beta-secretase cleavage site sequences include SEVKMDAEF (SEQ ID NO:3) and SEVNLDAEF (SEQ ID NO:4).

In some embodiments, the reporter molecule can be an enzyme, such as an alkaline phosphatase. In other embodiments, the reporter molecule can be a fluorophore, such as a green fluorescent protein (GFP). The reporter molecule can be a bioluminescent or a chemiluminescent polypeptide, such as an aequorin, an obelin, a mnemiopsin or a berovin. The chemiluminescent polypeptide can be luciferase.

One embodiment of the invention is a chimeric polypeptide having a first, a second and a third domain, wherein the first domain can be a Golgi retention signal peptide or an ER retention signal peptide, the third domain can be an alkaline phosphatase or a green fluorescent protein (GFP) reporter molecule, and the second domain can be a beta-secretase protease cleavage site located between the Golgi retention signal peptide and the reporter molecule.

In another aspect, the invention includes expression cassettes, expression vectors, and transformed host cells that contain the nucleic acids encoding the chimeric polypeptides of the invention described herein. The nucleic acid can be expressed in a cell-free system or a cell-based system to produce the polypeptide. The transformed host cell can be a bacterial cell, a mammalian cell, a yeast cell, an insect cell or a plant cell.

It is further contemplated that non-human animals and non-human transgenic animals may be used with the chimeric polypeptides and chimeric nucleic acids encoding the polypeptides. The chimeric nucleic acid encoding the polypeptide can be either exogenously added to the animal or it can be endogenous in the animal. The nucleic acid can be expressed in the cell of the animal to produce the polypeptide. Additionally or alternatively, the polypeptide can be introduced into the animal exogeneously. Examples of non-human transgenic animals contemplated by the invention include mice and rats, sheep, goats, pigs and the like.

Another aspect of the invention includes kits for the polypeptide and/or nucleic acids encoding the polypeptide and instructions for use. The instructions can include instructions on using the kit for measuring protease activity in vivo. The kits can further include a substrate for a bioluminescent polypeptide, chemiluminescent polypeptide or alkaline phosphatase.

Yet another aspect of the invention provides for methods of detecting protease activity by expressing the nucleic acid encoding the chimeric polypeptide or placing the chimeric polypeptide in a cell and detecting the amount of reporter molecule secreted by the cell. Such methods can also be used in intact non-human animals by expressing the nucleic acid encoding the chimeric polypeptide in the cells of the animal or providing the cells with the chimeric polypeptide and detecting the amount of reporter molecule secreted by the cell. The method can include administering to the non-human animal an expression vector or a recombinant virus that incorporates the chimeric nucleic acid. The method can include the use of transgenic non-human animals having chimeric nucleic acid.

Still another aspect of the invention provides for methods for identifying a modulator of protease activity including the steps of providing a test compound; expressing the chimeric nucleic acid in a cell or placing the chimeric polypeptide in a cell; detecting the amount of reporter molecule secreted by the cell; exposing the cell to the test compound and detecting the amount of reporter molecule secreted by the cell; and comparing the amount of reporter molecule secreted by the cell before exposure to the test compound to the amount of reporter molecule secreted by the cell after exposure to the test compound. The difference in amounts can identify the test compound as a modulator of protease activity. A decrease in the amount of reporter molecule secreted by the cell after exposure to the test compound can identify an inhibitor of the protease. An increase in the amount of reporter molecule secreted by the cell after exposure to the test compound can identify an activator of the protease.

The cell can be a bacterial cell, a mammalian cell, a yeast cell, an insect cell or a plant cell. The cell can be in a tissue culture media and detecting the amount of reporter molecule secreted by the cell includes measuring the amount of report molecule in the tissue culture media. The cell can also be a transgenic cell having and expressing the chimeric nucleic acid.

Alternatively, the cell can be part of an intact non-human animal. Detecting the amount of reporter molecule secreted by the cell includes taking a fluid sample from the animal and measuring the amount of reporter molecule in the fluid sample. The fluid sample can be a blood sample, a cerebral spinal fluid sample, a saliva sample or a urine sample. Detection of the amount of reporter molecule secreted by the cell can also be done by taking a tissue sample from the animal and measuring the amount of reporter molecule in the tissue sample. The tissue sample can be a biopsy sample.

In some embodiments, the protease cleavage site can be a secretase cleavage site. The secretase cleavage site can be a beta-secretase cleavage site or a gamma-secretase cleavage site. The beta-secretase cleavage site may have the sequence SEVKMDAEF (SEQ ID NO:3) or SEVNLDAEF (SEQ ID NO:4).

The reporter molecule can be a molecule that can be directly or by enzymatic reaction with a reagent generate a molecule that can be imaged by computer assisted tomography (CAT), magnetic resonance spectroscopy (MRS), magnetic resonance imaging (MRI), positron emission tomography (PET), single-photon emission computed tomography (SPECT), bioluminescence image (BLI) or equivalent. Examples of reporter molecules include a green fluorescent protein and an alkaline phosphatase. Reporter molecules can a bioluminescent or a chemiluminescent polypeptide. An example of a chemiluminescent polypeptide is luciferase. Other examples of a bioluminescent or a chemiluminescent polypeptide include aequorin, obelin, mnemiopsin and berovin.

In still another aspect, the invention provides for the screening of large numbers of compounds that may modulate protease activity with high throughput assays.

The details of one or more embodiments of the invention are set forth in the accompanying drawings and the description below. Other features, objects, and advantages of the invention will be apparent from the description, drawings, and claims.

All publications, patents, patent applications, GenBank sequences and ATCC deposits, cited herein are hereby expressly incorporated by reference for all purposes.

### DESCRIPTION OF DRAWINGS

FIG. 1 is a schematic describing an exemplary strategy for the noninvasive detection of a BACE, as described in detail in Example 1, below.

FIG. 2 is a schematic summary of data showing the expression of a BACE reporter or a BACE reporter having a Swedish mutation in COS cells alone or in the presence of exogenous BACE, as described in detail in Example 1, below.

FIG. 3 is a representation of a radiograph of a western blot of samples of tissue culture media to detect secreted molecules, as described in detail in Example 1, below.

FIG. 4 is a representation of a radiograph of a western blot of samples of cells extracts, as described in detail in Example 1, below.

FIG. 5 for a schematic summary of data showing KDEL dependent retention and BACE dependent secretion of alkaline phosphatase, as described in detail in Example 1, below.

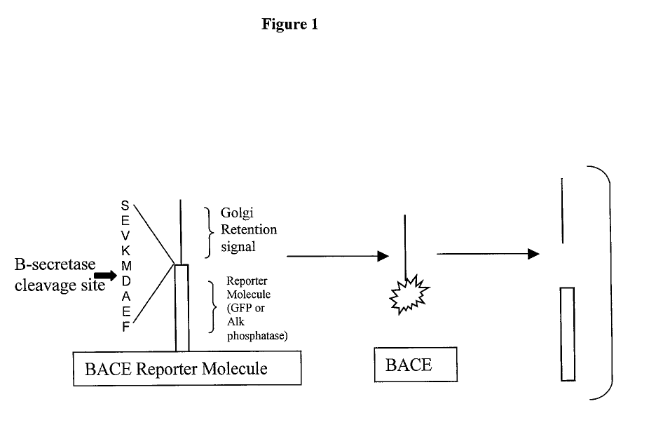

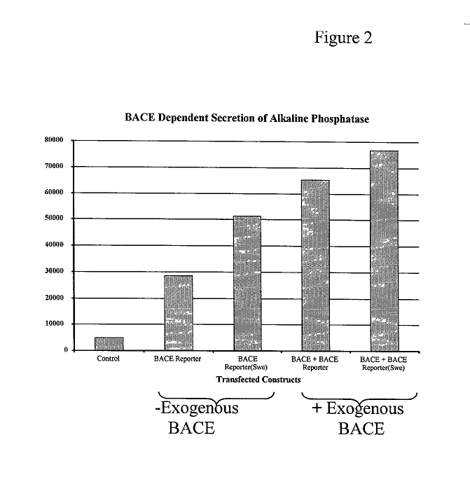

Like reference symbols in the various drawings indicate like elements.

### DETAILED DESCRIPTION

Precursor proteins are generally post-translationally modified in the endoplasmic reticulum (ER) and Golgi to an active secretable form. These posttranslational modifications can include proteolytic cleavage by proteases at specific cleavage sites. By taking advantage of proteases that are Golgi and/or endoplasmic reticulum (ER) resident proteinases and Golgi and/or ER retention signals, the present invention provides compositions and methods to detect and measure the activity of those proteases. Using these compositions, the invention also provides methods for detecting and screening for modulators of enzyme activity, e.g., for in vivo high throughput screening of inhibitors and activators of proteases.

One feature of the present invention is a construct that can be used to detect and measure protease activity. The construct can be a chimeric polypeptide or a nucleic acid encoding the chimeric polypeptide. The construct has a first domain that includes a Golgi and/or ER retention signal, a second domain that includes the cleavage site of the protease of interest, and a third domain that includes a reporter molecule. The reporter molecule is directed to, and can be retained, in the ER or Golgi because it is linked to a Golgi and/or an ER retention signal. However, in the presence of the protease of interest, the protease will cleave the chimeric peptide at its cleavage site, thereby releasing the reporter molecule and allowing it to be secreted out of the cell into the extracellular medium (e.g., tissue culture fluid, serum) where it can be detected and/or measured. The amount of reporter molecule in the extracellular medium can be correlated to the level of activity of the protease. The amount of reporter molecule in the extracellular medium also can be correlated to the level of activity of a putative protease inhibitor or activator.

For example, the protease of interest can be BACE, a beta-secretase (β-secretase). Thus, in one aspect, the construct comprises a Golgi retention signal, BACE cleavage site, and a reporter molecule (BACE reporter construct). Within the Golgi compartment, in the absence of BACE activity, the reporter molecule of the expressed BACE reporter construct cannot be released from the Golgi or ER retention signal. The BACE reporter construct is retained in the Golgi and, therefore, no amount of reporter molecule will be detected in the circulation (in transgenic animals) or the conditioned media (in tissue culture cells). However, in the presence of BACE activity, the BACE reporter construct is cleaved, thereby releasing the reporter molecule from the Golgi retention signal. By default, the reporter molecule is secreted from the cell and can be detected in the circulation or conditioned media.

BACE activity is of particular interest in the study of AD as it is a key enzyme in the production of the amyloid β-peptide. The amyloid β-peptide constitutes amyloid plaques, which are detected for the diagnosis of Alzheimer's disease. The amyloid β-peptide may be a causative agent for Alzheimer's disease. Thus, by detecting and/or measuring BACE activity, the methods of the invention provide a screening assay to identify compounds for the amelioration, detection and/or prevention of Alzheimer's disease. These compositions and methods can be used as cell based, in vivo high-throughput screening assays to identify a series of lead compounds.

In one aspect, the invention provides non-human animal (e.g., mouse) models that enable in vivo high-throughput screening to identify modulators of protease activities. These models also provide for testing of identified lead compounds for efficacy. In one aspect, the animal is a transgenic animal expressing a chimeric compound of the invention. The expression construct in the animal can be designed to be cell or tissue specific, or, constitutive or inducible. In one aspect, the animal has been engineered as a “knockout” of the protease to be whose activity is to be detected. For example, BACE has been identified as a critical enzyme in the production of the amyloid P-peptide. Mice lacking BACE are viable and have a major decrease in amyloid production. Thus, in one exemplary aspect, the invention provides a transgenic mouse comprising a chimeric nucleic acid of the invention.

The compositions and methods of the invention can also be practiced using non-transgenic animal models. In these aspects, a chimeric polypeptide of the invention is expressed in the animal by expression of vectors comprising a chimeric nucleic acid of the invention. For example, recombinant vectors, viruses, or naked DNA is used to transfect or infect cells in the living animal. The transfection or infection can be tissue specific, e.g., respiratory epithelium (inhalation) or liver (infusion in hepatic artery). The chimeric nucleic acid of the invention can be integrated in the chromosome or remain episomal. In another aspect, cells transformed with a chimeric nucleic acid of the invention and expressing a chimeric polypeptide of the invention are implanted in an animal.

Definitions

Unless defined otherwise, all technical and scientific terms used herein have the meaning commonly understood by a person skilled in the art to which this invention belongs. As used herein, the following terms have the meanings ascribed to them unless specified otherwise.

As used herein, the term “bioluminescence imaging” or “BLI” includes all bioluminescence, fluorescence or chemiluminescence or other photon detection systems and devices capable of detecting bioluminescence, fluorescence or chemiluminescence or other photon detection systems. Since light can be transmitted through mammalian tissues at a low level, bioluminescent and fluorescent proteins can be detected externally using sensitive photon detection systems; see, e.g., Contag (2000) Neoplasia 2:41-52; Zhang (1994) Clin. Exp. Metastasis 12: 87-92. The methods of the invention can be practiced using any such photon detection device, or variation or equivalent thereof, or in conjunction with any known photon detection methodology, including visual imaging. An exemplary photodetector device is an intensified charge-coupled device (ICCD) camera coupled to an image processor. See, e.g., U.S. Pat. No. 5,650,135. Photon detection devices are manufactured by, e.g., Xenogen (Alameda, Calif.) (the Xenogen IVISTM imaging system); or, Hamamatsu Corp., Bridgewater, N.J.

As used herein, a “computer assisted tomography (CAT)” or a “computerized axial tomography (CAT)” incorporates all computer-assisted tomography imaging systems or equivalents and devices capable of computer assisted tomography imaging. The methods of the invention can be practiced using any such device, or variation of a CAT device or equivalent, or in conjunction with any known CAT methodology. See, e.g., U.S. Pat. Nos. 6,151,377; 5,946,371; 5,446,799; 5,406,479; 5,208,581; 5,109,397. Animal imaging modalities are also included, such as MICROCATTM (ImTek, Inc., Knoxville, Tenn.).

As used herein, “positron emission tomography imaging (PET)” incorporates all positron emission tomography imaging systems or equivalents and all devices capable of positron emission tomography imaging. The methods of the invention can be practiced using any such device, or variation of a PET device or equivalent, or in conjunction with any known PET methodology. See, e.g., U.S. Pat. Nos. 6,151,377; 6,072,177; 5,900,636; 5,608,221; 5,532,489; 5,272,343; 5,103,098. Animal imaging modalities are included, e.g. micro-PETs (Corcorde Microsystems, Inc.).

As used herein, “single-photon emission computed tomography (SPECT) device” incorporates all single-photon emission computed tomography imaging systems or equivalents and all devices capable of single-photon emission computed tomography imaging. The methods of the invention can be practiced using any such device, or variation of a SPECT device or equivalent, or in conjunction with any known SPECT methodology. See, e.g., U.S. Pat. Nos. 6,115,446; 6,072,177; 5,608,221; 5,600,145; 5,210,421; 5,103,098. Animal imaging modalities are also included, such as micro-SPECTs.

As used herein, “magnetic resonance imaging (MRI) device” incorporates all magnetic resonance imaging systems or equivalents and all devices capable of magnetic resonance imaging. The methods of the invention can be practiced using any such device, or variation of an MRI device or equivalent, or in conjunction with any known MRI methodology. In magnetic resonance methods and apparatus a static magnetic field is applied to a tissue or a body under investigation in order to define an equilibrium axis of magnetic alignment in a region of interest. A radio frequency field is then applied to that region in a direction orthogonal to the static magnetic field direction in order to excite magnetic resonance in the region. The resulting radio frequency signals are detected and processed. The exciting radio frequency field is applied. The resulting signals are detected by radio-frequency coils placed adjacent the tissue or area of the body of interest. See, e.g., U.S. Pat. Nos. 6,151,377; 6,144,202; 6,128,522; 6,127,825; 6,121,775; 6,119,032; 6,115,446; 6,111,410; 602,891; 5,555,251; 5,455,512; 5,450,010; 5,378,987; 5,214,382; 5,031,624; 5,207,222; 4,985,678; 4,906,931; 4,558,279. MRI and supporting devices are manufactured by, e.g., Bruker Medical GMBH; Caprius; Esaote Biomedica (Indianapolis, Ind.); Fonar; GE Medical Systems (GEMS); Hitachi Medical Systems America; Intermagnetics General Corporation; Lunar Corp.; MagneVu; Marconi Medicals; Philips Medical Systems; Shimadzu; Siemens; Toshiba America Medical Systems; including imaging systems, by, e.g., Silicon Graphics. Animal imaging modalities are also included, such as micro-MRIs.

As used herein, the terms “computer” and “processor” are used in their broadest general contexts and incorporate all such devices. The methods of the invention can be practiced using any computer/processor and in conjunction with any known software or methodology. For example, a computer/processor can be a conventional general-purpose digital computer, e.g., a personal “workstation” computer, including conventional elements such as microprocessor and data transfer bus. The computer/processor can further include any form of memory elements, such as dynamic random access memory, flash memory or the like, or mass storage such as magnetic disc optional storage.

As used herein, “bioluminescent” and “chemiluminescent” polypeptides include all known polypeptides known to be bioluminescent or chemiluminescent, or, acting as enzymes on a specific substrate (reagent), can generate (by their enzymatic action) a bioluminescent or chemiluminescent molecule. They include, e.g., isolated and recombinant luciferases, aequorin, obelin, mnemiopsin, berovin and variations thereof and combinations thereof, as discussed in detail, below. In some aspects, the bioluminescent or chemiluminescent molecules are enzymes that act on a substrate that reacts with the reagent in situ to generate a molecule that can be imaged. The substrate can be administered before, at the same time (e.g., in the same formulation), or after administration of the chimeric polypeptide (including the enzyme).

The term “nucleic acid” or “nucleic acid sequence” refers to a deoxy-ribonucleotide or ribonucleotide oligonucleotide, including single- or double-stranded, or coding or non-coding (e.g., “antisense”) forms. The term encompasses nucleic acids, i.e., oligonucleotides, containing known analogues of natural nucleotides. The term also encompasses nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156.

The term “expression cassette” refers to any recombinant expression system for the purpose of expressing a nucleic acid sequence of the invention in vitro or in vivo, constitutively or inducibly, in any cell, including, in addition to mammalian cells, insect cells, plant cells, prokaryotic, yeast, fungal or mammalian cells. The term includes linear or circular expression systems. The term includes all vectors. The cassettes can remain episomal or integrate into the host cell genome. The expression cassettes can have the ability to self-replicate or not, i.e., drive only transient expression in a cell. The term includes recombinant expression cassettes that contain only the minimum elements needed for transcription of the recombinant nucleic acid.

As used herein the terms “polypeptide,” “protein,” and “peptide” are used interchangeably and include compositions of the invention that also include “analogs,” or “conservative variants” and “mimetics” (e.g., “peptidomimetics”) with structures and activity that substantially correspond to the polypeptides of the invention, including the chimeric polypeptide comprising a bioluminescent or chemiluminescent polypeptide, or a heterologous kinase, and a silencing moiety, and an endogenous protease cleavage motif positioned between the first and third domains. Thus, the terms “conservative variant” or “analog” or “mimetic” also refer to a polypeptide or peptide which has a modified amino acid sequence, such that the change(s) do not substantially alter the polypeptide's (the conservative variant's) structure and/or activity (e.g., binding specificity), as defined herein. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity. Conservative substitution tables providing functionally similar amino acids are well known in the art. For example, one exemplary guideline to select conservative substitutions includes (original residue followed by exemplary substitution): ala/gly or ser; arg/lys; asn/gln or his; asp/glu; cys/ser; gln/asn; gly/asp; gly/ala or pro; his/asn or gln; ile/leu or val; leu/ile or val; lys/arg or gln or glu; met/leu or tyr or ile; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W); (see also, e.g., Creighton (1984) Proteins, W. H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations.”

The terms “mimetic” and “peptidomimetic” refer to a synthetic chemical compound that has substantially the same structural and/or functional characteristics of the polypeptides of the invention (e.g., ability to be specifically recognized and cleaved by enzymes, including proteases). The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetics' structure and/or activity. As with polypeptides of the invention which are conservative variants, routine experimentation will determine whether a mimetic is within the scope of the invention, i.e., that its structure and/or function is not substantially altered. Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclo-hexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH2- for —C(═O)—NH—), aminomethylene (CH2-NH), ethylene, olefin (CH═CH), ether (CH2-O), thioether (CH2-S), tetrazole (CN4-), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY). A polypeptide can also be characterized as a mimetic by containing all or some non-natural residues in place of naturally occurring amino acid residues; non-natural residues are well described in the scientific and patent literature.

Golgi and ER Retention Signal Peptides

The invention provides chimeric nucleic acids encoding polypeptides comprising Golgi and/or ER retention signal peptides and chimeric polypeptides comprising Golgi and/or ER retention signal peptides. Any Golgi and/or ER retention signal can be used and Golgi and/or ER retention signal peptides are well known in the art. For example, in one aspect, the Golgi and/or ER retention signal peptide is a Golgi/ER signal peptide from a Golgi or ER resident enzyme.

Methods for determining the Golgi and ER retention signal motifs, e.g., of a Golgi resident enzyme, are well known in the art and can be determined by routine screening. See, e.g., Opat (2001) Biochimie 83:763-773; Pelham (2000) Traffic 1:191-192; Munro (1998) Trends Cell Biol 8:11-15; U.S. Pat. Nos. 5,776,772; 5,578,466, 5,541,083; 5,032,519. For example, the amount of mutant protein lacking the putative Golgi and/or ER retention signal peptide that is secreted by the cell is compared to the amount of protein having the putative Golgi and/or ER retention signal peptide is secreted by the cell.

In one aspect, the ER retention signal comprises a KDEL (SEQ ID NO:1) signal. See, e.g., Pap (2001) Exp. Cell Res. 265:288-293; Majoul (2001) Dev. Cell 1:139-153. The Golgi retention signal can also comprise a KKAA (SEQ ID NO:5) signal. The KDEL (SEQ ID NO:1) and KKAA (SEQ ID NO:5) motifs act as a mechanism for the retention of proteins in the endoplasmic reticulum (ER). See, e.g., Dogic (2001) Eur. J. Cell Biol. 80:151-155; Andersson (1999) J. Biol. Chem. 274:15080-15084. Other Golgi and/or ER retention signal peptides can include HDEL (SEQ ID NO:6), DDEL (SEQ ID NO:7), ADEL (SEQ ID NO:8), SDEL (SEQ ID NO:9), RDEL (SEQ ID NO:10), KEEL (SEQ ID NO:11), QEDL (SEQ ID NO:12), HIEL (SEQ ID NO:13), HTEL (SEQ ID NO:14), KQDL (SEQ ID NO:15), and PTEL (SEQ ID NO:16). See, e.g., U.S. Pat. Nos. 5,747,660; and 5,578,466. A peptide sequence within the Leu/Ile-rich region of the human Ca(2+)-binding EF-hand/leucine zipper protein NEFA (SEQ ID NO:2) can also be used as a Golgi retention motif. See, e.g., Nesselhut (2001) FEBS Lett. 509:469-475. Furthermore, synthetic Golgi and ER retention signals can be designed and incorporated into the compositions and methods of the invention.

In one aspect, a peptide sequence of a Golgi glycosyltransferase is used as a Golgi retention motif. For example, Golgi retention signal peptides from a N-acetylglucosaminyltransferase I (GlcNAcTI), a beta 1,4-galactosyltransferase (GaIT), an alpha 2,6-sialytransferase (ST), a beta-galactoside alpha 2,6-sialyltransferase (ST) and a Nacetylglucosaminyltransferase I (NT) are used. Golgi glycosyltransferase Golgi retention signal motifs are well known in the art. See, e.g., Colley (1997) Glycobiology 7:1-13; Tang (1995) Eur. J. Cell Biol. 66:365-374. See also U.S. Pat. Nos. 5,776,772; 5,541,083; 5,032,519, which describe the identifying and cloning of Golgi glycosyltransferase Golgi retention signal peptides.

Proteases and Protease Cleavage Sites

The invention provides chimeric nucleic acids encoding polypeptides comprising protease cleavage motif and chimeric polypeptides comprising a protease cleavage motif. The protease cleavage site can be that of the protease whose activity is being detected or measured. The protease of interest can be any proteolytic enzyme, including those that are ER or Golgi resident enzymes. Thus, any protease cleavage recognition site can be used. Protease cleavage recognition site can also be non-natural, engineered enzyme cleavage sites.

Examples of proteases, and their corresponding protease cleavage sites include, but are not limited to, subtilisn-like proteases, members of the Kex2 (or kexin) gene family, such as prohormone convertases (e.g., PC1, also known as PC3 and SPC3, PC2, furin, PACE4, PC4, PC5, and PC7), and Subtilisin-Kexin-Isozyme (e.g., SKI-1).

Any of the many known protease cleavage motifs can be used in the chimeric polypeptide of the invention. For example, the preferred cleavage site for furin is Arg X Lys/Arg Arg (SEQ ID NO:17). Alternatively, entirely synthetic protease cleavage motifs can be devised and incorporated. In general, these proteolytic enzymes cleave proproteins at selected sites composed of single or paired basic amino acids. They specialize in cleavage at basic residues (usually arginines) within the general motif (Arg/Lys)-(X)n-Arg***where N=0, 2, 4, or 6 and X is any amino acid. These enzymes can also cleave at non-basic sites, such as C-terminal to Ala, Ser, Thr, Met, Val, and Leu. Examples of cleavage sites are provided in Seidah, et al. Brain Research Interactive 848 (1999) 45-62.

Endogenous protease cleavage recognition domains can also be derived from matrix metalloproteinase (MMP) enzymes (see, e.g., U.S. Pat. Nos. 6,140,099; 6,114,568; 6,093,398; 5,595,885); secretins; gamma-secretase associated with Alzheimer's disease (see, e.g., Zhang (2000) Nat. Cell Biol. 2:463-465); calpain proteases (also associated with Alzheimer's disease, see e.g., Nath (2000) Biochem. Biophys. Res. Commun 274:16-21; Wang (2000) Trends Neurosci. 23:20-26). Other examples include cleavage site recognized by thrombin, H64A subtilisin, and enterokinase described by Forsberg (1992) J. Protein Chem. 11:201-211. Humphreys (2000) Protein Eng. 13:201-206, described an improved efficiency of the site-specific copper (II) ion-catalyzed protein cleavage peptide sequence (N)DKTH(C) (SEQ ID NO:18) effected by mutagenesis of cleavage site. Various virus-specified protease cleavage recognition sites are described in U.S. Pat. No. 4,952,493.

The protease cleavage motif can be positioned between the first and third domains of the chimeric polypeptide. In one aspect, the protease cleavage motif can be flanked by a “spacer” on one or both sides (i.e., a spacer is between the cleavage motif and either or both the signaling domain and the reporter domain, e.g., the bioluminescent or chemiluminescent polypeptide domain. The spacer can be, e.g., a poly-glycine moiety. Other “spacers” are known in the art; for example, to improve site-specific cleavage of a methionyl porcine growth hormone [[Met1]-pGH(1-46)-IGF-11] fusion protein by the enzyme H64A subtilisin, Polyak (1997) Protein Eng. 10:615-619, introduced a series of flexible, unstructured spacer peptides N-terminal to the cleavage site.

Proteases useful to the present invention include secretases related to Alzheimer's dementia. These secretases are described, e.g., in U.S. Pat. Nos. 6,313,268; 6,245,884; 6,221,645; 5,942,400; and 5,744,346. They include alpha, beta, and gamma secretases. In alternative aspects, beta- and gamma-secretase activities, which are known to be ER or Golgi resident proteases, are detected and measured. BACE is a known beta-secretase involved in AD.

Reporter Molecules

The invention provides chimeric nucleic acids encoding reporter molecule polypeptides and chimeric polypeptides comprising a reporter molecule. Any reporter molecule, i.e., any molecule that can directly or indirectly generate a detectable signal, can be used in the compositions and methods of the invention. Reporter molecules encoded by the chimeric nucleic acids of the invention can be polypeptides that are detectable, e.g., because they have an epitope detectable by an antibody (e.g., a polyhistidine, FLAG and the like) or other ligand binding moiety (e.g., a receptor), or, because they have enzymatic activity that can generate a detectable signal. Alternatively, reporter molecules can be any detectable molecule attached to a chimeric polypeptide of the invention, including, e.g., radioactive molecules or isotopes, peptide or inorganic antibody epitopes, and the like.

Exemplary reporter molecules include E. coli beta-galactosidase (An, G., Hidaka, K., Siminovitch, L., Mol. Cell. Biol. 2, 1628-1632 (1982)), xanthine-guanine phosphoribosyl transferase (Chu, G., Berg, P., Nucleic Acid Res. 13, 2921-2930 (1985)), galactokinase (Schumperli, D., Howard, B., Rosenberg, M., Proc. Natl. Acad. Sci. USA 79, 257-261 (1982)), beta.-lactamase (Cartwright, C. P., Li, Y., Zhu, Y. S., Kang, Y. S., Tipper, D. J., Yeast 10, 497-508 (1994)), beta-tactamase (Zlokarnik, G., Negulescu, P. A., Knapp, T. E., Mere, L., Burres, N., Feng, L., Whitney, M., Roemer, K., and Tsien, R. Y., Science 279(5347), 84-88 (1998)), thymidine kinase (Searle, P., Stuart, G., Palmiter, R., Mol. Cell. Biol. 5, 1480-1489 (1985)), chloramphenicol acetyltransferase (Gorman, C., Moffat, L., Howard, B., Mol. Cell. Biol. 2, 1044-1051 (1982)), alkaline phosphatase (Berger, J., Hauber, J., Hauber, R., Geiger, R., Cullen, B., Gene 66, 1-10 (1988); Cullen, B., Malin, M., Methods Enzymol. 216, 362-368 (1992); Bronstein, I., BioTechniques 17, 172-178, (1994)), and urokinase-plasminogen activator (Yokoyama-Kobayashi, M., Sugano, S., Kato, T., Kato, S., Gene 163, 193-196 (1995), Zimmerman, M., Quigley, J. P., Ashe, B., Dorn, C., Goldfarb, R., Troll, W., Proc. Natl. Acad. Sci. USA 75, 750-753 (1978), Huseby, R. M., et al., Thrombosis Research 10, 679 (1977)).

Bioluminescent or Chemiluminescent Polypeptides

In one aspect, the report molecule comprises a bioluminescent or chemiluminescent polypeptide. As defined above, these polypeptides include enzymes that act on a specific reagent to generate a molecule that can be imaged (e.g., luciferase reacting with luciferin in situ). Once cleaved, the bioluminescent or chemiluminescent domain is “liberated” from its “silencer” to be used as a reporter in quantitative assays to non-invasively image the endogenous enzyme (e.g., protease) activity (the protease specific for the cleavage motif). Thus, it can be imaged that the chimeric polypeptide of the invention could comprise a reporter molecule that is separated from its silencer by the second domain (i.e., the cleavage site of the protease of interest). Thus, once the protease cleaves the chimeric polypeptide, the reporter molecule is liberated from the silencer as well as the Golgi and/or ER retention signal peptide. Alternatively, the reporter molecule and its silencer are not separated by the second domain and the reporter molecule is liberated from its silencer at some time after the protease of interest has cleaved the chimeric polypeptide separating the reporter molecule and its silencer from the Golgi and/or ER retention signal peptide. The kinase activity can be imaged in living animals using MRI, PET, SPECT and the like.

In alternative aspects, these polypeptides include, e.g., luciferase, aequorin, halistaurin, phialidin, obelin, mnemiopsin or berovin, or, equivalent photoproteins, and combinations thereof. The compositions and methods of the invention also include recombinant forms of these polypeptides as recombinant chimeric or “fusion” proteins, including chimeric nucleic acids and constructs encoding them. Methods of making recombinant forms of these polypeptides are well known in the art, e.g., luciferase reporter plasmids are described, e.g., by Everett (1999) J. Steroid Biochem. Mol. Biol. 70:197-201. Sala-Newby (1998) Immunology 93:601-609, described used of a recombinant cytosolic fusion protein of firefly luciferase and aequorin (luciferase-aequorin). The Ca2+-activated photoprotein obelin is described by, e.g., Dormer (1978) Biochim. Biophys. Acta 538:87-105; and, recombinant obelin is described by, e.g., Illarionov (2000) Methods Enzymol. 305:223-249. The photoprotein mnemiopsin is described by, e.g., Anctil (1984) Biochem J. 221:269-272. The monomeric Ca2+-binding protein aequorin is described by, e.g., Kurose (1989) Proc. Natl. Acad. Sci. USA 86:80-84; Shimomura (1995) Biochem. Biophys. Res. Communm 211:359-363. The aequorin-type photoproteins halistaurin and phialidin are described by, e.g., Shimomura (1985) Biochem J. 228:745-749. Ward (1975) Proc. Natl. Acad. Sci USA 72:2530-2534, describes the purification of mnemiopsin, aequorin and berovin. The recombinant bioluminescent or chemiluminescent chimeric polypeptides of the invention can be made by any method, see, e.g., U.S. Pat. No. 6,087,476, that describes making recombinant, chimeric luminescent proteins. U.S. Pat. Nos. 6,143,50; 6,074,859; 6,074,859, 5,229,285, describe making recombinant luminescent proteins. The bioluminescent or chemiluminescent activity of the chimeric recombinant polypeptides of the invention can be assayed, e.g., using assays described in, e.g., U.S. Pat. Nos. 6,132,983; 6,087,476; 6,060,261; 5,866,348; 5,094,939; 5,744,320. Various photoproteins that can be used in compositions of the invention are described in, e.g., U.S. Pat. Nos. 5,648,218; 5,360,728; 5,098,828.

Exemplary labels include, e.g., 32P, 35S, 3H, 14C, 125I, 131I; fluorescent dyes (e.g., Cy5™, Cy3™, FITC, rhodamine, lanthanide phosphors, Texas red), electrondense reagents (e.g. gold), enzymes, e.g., as commonly used in an ELISA (e.g., horseradish peroxidase, beta-galactosidase, luciferase, alkaline phosphatase), colorimetric labels (e.g. colloidal gold), magnetic labels (e.g. DynabeadsTM), biotin, dioxigenin, or haptens and proteins for which antisera or monoclonal antibodies are available. The label can be directly incorporated reporter molecule be detected, or it can be attached to a probe or antibody that hybridizes or binds to the reporter molecule target. A peptide can be made detectable by incorporating (e.g., into a nucleoside base) predetermined polypeptide epitopes recognized by a secondary reporter (e.g., leucine zipper pair sequences, binding sites for secondary antibodies, transcriptional activator polypeptide, metal binding domains, epitope tags). Label can be attached by spacer arms of various lengths to reduce potential steric hindrance or impact on other useful or desired properties. See, e.g., Mansfield (1995) Mol Cell Probes 9:145-156.

In vivo Bioluminescent Imaging

The invention provides compositions and methods for detecting the activity of proteases and screening for modulators of protease activity in vivo. Reporter molecules are released by cells with protease activity and detected and measured in the extracellular milieu, e.g., in extracellular tissue spaces, serum, blood, and the like. In one aspect, the reporter molecules are detected by bioluminescence imaging (BLI). In vivo Bioluminescent Imaging (BLI) is an imaging modality, see e.g., Contag (2000) Neoplasia 2:41-52. In one aspect, the reporter molecule is a photoprotein (i.e., an optical reporter), such as luciferase from the firefly. It can be detected using a sensitive photon detection system. The number of photons emitted from cells expressing the photoprotein (e.g., luciferase) can be quantitatively detected and overlayed (projected) onto a visual picture of the animal (including humans). This imaging approach provides a two-dimensional image data set and thus provides some spatial information as to the origin of the signal within the animal.

Polypeptides and Peptides

The invention provides a chimeric polypeptide comprising a first domain comprising a Golgi and/or ER retention signal peptide, a third domain comprising a reporter molecule and a second domain comprising at least one protease cleavage motif positioned between the first and third domains. As noted above, the term polypeptide includes peptides and peptidomimetics, etc. Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art.

Polypeptide and peptides of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer). Where the desired sequences are relatively short, the polypeptide may be synthesized as a single contiguous polypeptide. Where larger molecules are desired, the polypeptide can be synthesized separately as units and then fused by condensation of the amino terminus of one peptide unit with the carboxyl terminus of the other peptide unit, thereby forming a peptide bond. The skilled artisan will recognize that individual synthetic residues and polypeptides incorporating mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Polypeptides incorporating mimetics can also be made using solid phase synthetic procedures, as described, e.g., by Di Marchi, et al., U.S. Pat. No. 5,422,426. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234. Modified peptides of the invention can be further produced by chemical modification methods, see, e.g., Belousov (1997) Nucleic Acids Res. 25:3440-3444; Frenkel (1995) Free Radic. Biol. Med. 19:373-380; Blommers (1994) Biochemistry 33:7886-7896.

Peptides and polypeptides of the invention can also be synthesized and expressed as chimeric or “fusion” proteins with one or more additional domains linked thereto for, e.g., to more readily isolate a recombinantly synthesized peptide, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) to the chimeric polypeptide of the invention can be useful to facilitate purification. For example, an expression vector can include the chimeric polypeptide-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-14). The histidine residues facilitate detection and purification while the enterokinase cleavage site provides a means for purifying the polypeptide from the remainder of the fusion protein.

Nucleic Acids and Expression Vectors

This invention provides nucleic acids encoding the chimeric polypeptides of the invention and expression cassettes, e.g., vectors, plasmids, recombinant viruses, and the like. As the genes and expression cassettes (e.g., vectors) of the invention can be made and expressed in vitro or in vivo, the invention provides for a variety of means of making and expressing these genes and vectors. One of skill will recognize that desired phenotypes can be obtained by modulating the expression or activity of the genes and nucleic acids (e.g., promoters) within the expression cassettes of the invention. Any of the known methods described for increasing or decreasing expression or activity can be used for this invention. The invention can be practiced in conjunction with any method or protocol known in the art, which are well described in the scientific and patent literature.

The chimeric peptide can be prepared using recombinant methods. Generally this involves creating a DNA sequence that encodes the chimeric polypeptide, placing the DNA in an expression cassette under the control of a particular promoter, expressing the polypeptide in a host, isolating the expressed polypeptide and, if required, renaturing the peptide. Because the polypeptide of the invention are not found in nature, recombinant production generally involves synthesis of a nucleic acid that encodes the polypeptide. DNA encoding the polypeptides of this invention can be prepared by any suitable method including, for example, cloning and restriction of appropriate sequences or direct chemical synthesis by methods such as the phosphotriester method of Narang et al. Meth. Enzymol. 68: 90-99 (1979); the phosphodiester method of Brown et al., Meth. Enzymol. 68: 109-151 (1979); the diethylphosphoramidite method of Beaucage et al., Tetra. Lett., 22:1859-1862 (1981); and the solid support method of U.S. Pat. No. 4,458,066.

These nucleic acids can be synthesized in vitro by well-known chemical synthesis techniques, as described in, e.g., Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066. For example, chemical synthesis can be used to produce a single stranded oligonucleotide. This can be converted into double stranded DNA by hybridization with a complementary sequence, or by polymerization with a DNA polymerase using the single strand as a template. One of skill would recognize that while chemical synthesis of DNA may be limited to sequences of about 100 bases (subsequences), longer sequences may be obtained by the ligation of the shorter subsequences. Alternatively, the subsequences can be cloned and the appropriate subsequences cleaved using appropriate restriction enzymes. The fragments can then be ligated to produce the desired DNA sequence.

The nucleic acid sequences of the invention and other nucleic acids used to practice this invention, whether RNA, cDNA, genomic DNA, expression cassettes, vectors, viruses or hybrids thereof, may be isolated from a variety of sources, genetically engineered, amplified, and/or expressed recombinantly. Any recombinant expression system can be used, including, bacterial, mammalian, yeast, insect and plant cell expression systems.

Techniques for the manipulation of nucleic acids, such as, e.g., generating mutations in sequences, subcloning, labeling probes, sequencing, hybridization and the like are well described in the scientific and patent literature, see, e.g., Sambrook, ed., Molecular Cloning: a Laboratory Manual (2nd ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular BIOLOGY: HYBRIDIZATION WITH NUCLEIC ACID PROBES, Part I. Theory and Nucleic Acid Preparation, Tijssen, ed. Elsevier, N.Y. (1993).

Transformed Cells and Cell Lines

This invention provides cells comprising nucleic acids encoding the chimeric polypeptides of the invention. The cells can be used to screen for protease activity and for modulators of that activity in cell culture or in an intact animal, e.g., by implantation. Alternatively, the cells can be used merely to produce the recombinant fusion proteins of the invention, which can be used for in vitro or in vivo protease or modulator screening assays.

The nucleic acid sequences encoding the receptor peptides can be expressed in a variety of host cells including any eukaryotic cell, prokaryotic cell, or multicellular organism. Examples of eukaryotic cells include, but are not limited to, Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus. Examples of yeast cells include, but are not limited to, Saccharomyces cerevisiae, Schizosaccharomyces pombe, and Pichia pastoris. Examples of higher eukaryotic cells include, but are not limited to, COS, CHO, CV-1, HeLa, amphibian cells, such as Xenopus egg cell, and myeloma cell lines. Insect cells may also be utilized as host cells in the method of the present invention. See, e.g. Miller et al. (Genetic Engineering (1986) 8:277-298, Plenum Press) and references cited therein.

The recombinant peptide gene can be operably linked to appropriate expression control sequences for each host. For E. coli, this includes a promoter such as the T7, trp, or lambda promoters, a ribosome binding site and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences.

The plasmids of the invention can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Transgenic Non-Human Animals

The invention provides transgenic non-human animals, e.g., goats, rabbits, sheep, pigs, cows, rats and mice, comprising the chimeric nucleic acids of the invention. These animals can be used, e.g., as in vivo models to study protease activity, or, as models to screen for modulators of protease activity in vivo. In alternative aspects, the activity of an enzyme capable of cleaving an endogenous protease cleavage domain on an in vivo produced chimeric polypeptide is measured by BLI, PET, MRI, etc. As demonstrated in Example 1, below, such transgenic non-human animals are excellent models for imaging protease activity in vivo. The transgenic or modified animals of the invention can be administered putative modulators of protease activity and subjected to an imaging methodology, e.g., BLI, PET or MRI.

The coding sequences for the chimeric polypeptides can be designed to be constitutive, or, under the control of tissue-specific, developmental-specific or inducible transcriptional regulatory factors. Transgenic non-human animals can be designed and generated using any method known in the art; see, e.g., U.S. Pat. Nos. 6,211,428; 6,187,992; 6,156,952; 6,118,044; 6,111,166; 6,107,541; 5,959,171; 5,922,854; 5,892,070; 5,880,327; 5,891,698; 5,639,940; 5,573,933; 5,387,742; 5,087,571, describing making and using transformed cells and eggs and transgenic mice, rats, rabbits, sheep, pigs and cows. See also, e.g., Pollock (1999) J. Immunol. Methods 231:147-157, describing the production of recombinant proteins in the milk of transgenic dairy animals; Baguisi (1999) Nat. Biotechnol. 17:456-461, demonstrating the production of transgenic goats. U.S. Pat. No. 6,211,428, describes making and using transgenic non-human mammals which express in their brains a nucleic acid construct comprising a DNA sequence. U.S. Pat. No. 5,387,742, describes injecting cloned recombinant or synthetic DNA sequences into fertilized mouse eggs, implanting the injected eggs in pseudo-pregnant females, and growing to term transgenic mice whose cells express proteins related to the pathology of Alzheimer's disease. U.S. Pat. No. 6,187,992, describes making and using a transgenic mouse whose genome comprises a disruption of the gene encoding amyloid precursor protein (APP).

As discussed above, “knockout animals” can also be used to practice the methods of the invention. For example, in one aspect, the transgenic or modified animals of the invention comprise a “knockout animal,” e.g., a “knockout mouse,” engineered not to express or to be unable to express the protease to be detected using the composition of the invention.

Kits

The invention provides kits comprising the compositions, e.g., as pharmaceutical compositions, nucleic acids, expression cassettes, vectors, cells of the invention, to image the activity of endogenous enzymes. The kits also can contain instructional material teaching methodologies, e.g., how and when to administer the pharmaceutical compositions, how to apply the compositions and methods of the invention to imaging systems, e.g., computer assisted tomography (CAT), magnetic resonance spectroscopy (MRS), magnetic resonance imaging (MRI), positron emission tomography (PET), single-photon emission computed tomography (SPECT) or bioluminescence imaging (BLI). Kits containing preparations (e.g., chimeric polypeptides, expression cassettes, vectors, nucleic acids) can include directions as to indications, dosages, routes and methods of administration, and the like.

High Throughput Screening

The invention provides for methods for screening large numbers of compounds as modulators of protease activity. The compositions and methods of the invention can be used quickly and efficiently as “high throughput screening (HTS)” methods. High throughput screening methods involve providing a library containing a large number of potential therapeutic compounds (“candidate compounds”) that may be modulators of protease activity. These libraries are called “combinatorial chemical libraries” which can be screened using one or more assays of the invention, as described herein, to identify those library members (particular chemical species or subclasses) that display a desired characteristic activity, e.g., modulation of protease activity. The compounds thus identified can serve as conventional “lead compounds.” Once a lead compound is identified, new chemical entities with useful properties can be generated by creating variants of the lead compound. Assays of the invention can be used to evaluate the property and activity of the variant compounds. The lead compounds can themselves be used as potential or actual therapeutics.

Because of their ability to test large numbers quickly and efficiently, high throughput screening (HTS) methods can replace conventional lead compound identification methods. Thus, using the compositions and methods of the invention, it is possible to screen up to several thousand different modulators in a single day. For example, each well of a microtiter plate can be used to run a separate assay against a selected potential modulator. Thus, a single standard microtiter plate can assay about 100 (96) modulators. If 1536 well plates are used, then a single plate can easily assay up to 1500 different compounds.

The high through-put screening methods of the invention may also be automated. High through-put systems are available that typically include any one or more of the following: robotic armature which transfers fluids from a source to a destination, controller which controls the robotic armature, label/reporter detector, data storage unit, an assay component such as microtiter plates comprising wells for running the assays and testing the compounds, and a plate conveyor system. See, e.g., U.S. Pat. Nos. 6,306,659 and 6,207,391.

Any high throughput screening systems can be used in practicing the invention; many are commercially available (see, e.g., LEADseeker™ Amersham Pharmacia Biotech, Piscataway, N.J.; PE Biosystem FMATTM 8100 HTS System Automated, PE Biosystem, Foster City, Calif.; Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for the various high throughput assays. In addition, many product and service providers are available to assist in designing assays and utilizing high throughput screening systems, e.g., Alanex, La Jolla, Calif.; Amersham Biosciences, Buckinghamshire, UK; Applied Biosystems, Foster City, Calif.; Argones, Inc. Charlottesville, Va.; and BioMed Tech, Tampa, Fla.

Combinatorial Chemical Libraries

The compositions and methods of the invention are used to screen combinatorial chemical libraries for protease inhibitors to identify compounds that modulate, i.e., increase or decrease, protease activity. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, the systematic, combinatorial mixing of 100 interchangeable chemical building blocks results in the theoretical synthesis of 100 million tetrameric compounds or 10 billion pentameric compounds (see, e.g., Gallop et al. (1994) 37(9): 1233-1250).

Preparation and screening of combinatorial chemical libraries are well known to those of skill in the art, see, e.g., U.S. Pat. Nos. 6,096,496; 6,075,166; 6,054,047; 6,004,617; 5,985,356; 5,980,839; 5,917,185; 5,767,238. Such combinatorial chemical libraries include, but are not limited to, peptide libraries (see, e.g., U.S. Pat. No. 5,010,175; Furka (1991) Int. J. Pept. Prot. Res., 37: 487-493, Houghton et al. (1991) Nature, 354: 84-88). Other chemistries for generating chemical diversity libraries include, but are not limited to: peptoids (see, e.g., WO 91/19735), encoded peptides (see, e.g., WO 93/20242), random bio-oligomers (see, e.g., WO 92/00091), benzodiazepines (see, e.g., U.S. Pat. No. 5,288,514), diversomers such as hydantoins, benzodiazepines and dipeptides (see, e.g., Hobbs (1993) Proc. Nat. Acad. Sci. USA 90: 6909-6913), vinylogous polypeptides (see, e.g., Hagihara (1992) J. Amer. Chem. Soc. 114: 6568), nonpeptidal peptidomimetics with a Beta-D-Glucose scaffolding (see, e.g., Hirschmann (1992) J. Amer. Chem. Soc. 114: 9217-9218), analogous organic syntheses of small compound libraries (see, e.g., Chen (1994) J. Amer. Chem. Soc. 116: 2661), oligocarbamates (see, e.g., Cho (1993) Science 261:1303), and/or peptidyl phosphonates (see, e.g., Campbell (1994) J. Org. Chem. 59: 658). See also Gordon (1994) J. Med. Chem. 37:1385; for nucleic acid libraries, peptide nucleic acid libraries, see, e.g., U.S. Pat. No. 5,539,083; for antibody libraries, see, e.g., Vaughn (1996) Nature Biotechnology 14:309-314; for carbohydrate libraries, see, e.g., Liang et al. (1996) Science 274: 1520-1522, U.S. Pat. No. 5,593,853; for small organic molecule libraries, see, e.g., for isoprenoids U.S. Pat. No. 5,569,588; for thiazolidinones and metathiazanones, U.S. Pat. No. 5,549,974; for pyrrolidines, U.S. Pat. Nos. 5,525,735 and 5,519,134; for morpholino compounds, U.S. Pat. No. 5,506,337; for benzodiazepines U.S. Pat. No. 5,288,514.

Devices for the preparation of combinatorial libraries are commercially available (see, e.g., U.S. Pat. No. 6,045,755; 5,792,431; 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). A number of robotic systems have also been developed for solution phase chemistries. These systems include automated workstations, e.g., like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., CbemStar, Ltd, Moscow, RU, 3D Pharmaceuticals, Exton, Pa., Martek Biosciences, Columbia, Md., etc.).

High Throughput Assays of Chemical Libraries

The compositions and methods of the invention can be used in both cell-based and cell-free assays For example, in a cell based assay, cells having a nucleic acid of the invention and expressing the chimeric polypeptide are placed in test wells. Because of the Golgi and/or ER retention signal peptide, the polypeptide is retained in the Golgi/ER. The protease of interest, however, cleaves the chimeric polypeptide at its cleaving site and releases the reporter molecule from the Golgi and/or ER retention signal peptide. Once freed, the reporter molecule can then be secreted out of the cell into the surrounding media. The media in the well is tested to measure the level of reporter molecule present. The test compound can then be added to the test well. If the test compound modulates the activity of the protease, it will become evident by the subsequent increase or decrease of the level of reporter molecules in the media.

The objective is to detect a measurable change in the activity of the protease in the presence of a test compound. The level of activity is measured by the amount of reporter molecules secreted into the media. The measurable change will vary depending on the assay system and the method of measuring the reporter molecule. The present invention encompasses any difference between the pre and post test compound levels of reporter molecule secretion, where the difference is greater than expected due to random statistical variation. Any amount of change in activity of the protease identifies a compound as a modulator of protease activity.

In any assay, controls may be used to ensure that the assay is working properly. An assay may contain negative controls, wherein the control well contains all the reagents, except for a test compound, and are run under the same conditions as the test wells. No change in the control well should be detected, indicating that the system is running properly. Assays may also contain positive controls, wherein the control well contains all the reagents, except a compound whose effect is known is added instead of a test compound, and run under the same conditions. The activity in the well should be consistent with what is expected from the known compound, indicating that the system is running properly.

In vitro conditions for beta-secretase activity assays, e.g., screening for inhibitors of the enzyme, are well known in the art. See, e.g., U.S. Pat. Nos. 6,333,167; 6,329,163; 6,313,268; 6,245,884; 5,942,400; 5,744,346; which describe assays and conditions for beta-secretase activity assays. The invention also provides for high throughput whole cell assay screening systems. See, e.g., U.S. Pat. No. 5,763,198.

### EXAMPLES

The following example is offered to illustrate, but not to limit the claimed invention.

### Example 1

### Non-Invasive Reporting of Protease Activity

The following example demonstrates use of the compositions and methods of the invention to report on the activity of enzymes in vivo non-invasively.

Expression of a BACE reporter or a BACE reporter having a Swedish mutation in COS cells alone or in the presence of exogenous BACE were studied. A BACE reporter construct was created, wherein the reporter was alkaline phosphatase.

The reporter was constructed using standard recombinant DNA methodology. A secreted form of alkaline phosphatase (AP) or GFP were used as reporters. These reporters were constructed such that they contained the BACE cleavage site SEVKAMDAEF (SEQ ID NO:3) or SEVNLDAEF (SEQ ID NO:4) followed by the KDEL sequence at the carboxy-terminus. The presence of the KDEL sequence at the carboxyl-terminus on each of these reporters resulted in ER retention of the reporter molecule. When BACE or a BACE-like protease cleaved the SEVKAMDAEF (SEQ ID NO:3) or SEVNLDAEF (SEQ ID NO:4) sequence, the reporter molecule would be free to exit the ER and thus show up extracellularly in the media and could therefore be measured.

The above recombinant DNA molecules contained within a standard expression vector were transiently transfected into COS cells (African Green Monkey Kidney cells) or neuro-2 cells (undifferentiated neuronal cells) using lipofection. 48 hrs after transfection, the presence of the recombinant protein was measured in cell extracts (FIG. 4) or in the conditioned media (FIG. 3) by western blot analysis using the appropriate antibody (anti-AP or anti-GFP) as well as using a biological assay for AP (chemiluminescence) or GFP (fluorescence). ER retention of the reporter wherein the SEVKAMDAEF (SEQ ID NO:3) sequence preceded the KDEL sequence resulted in little or no detectable reporter activity in the media of transfected cells (see FIG. 3, lane 3). In contrast, when the SEVNLDAEF (SEQ ID NO:4) sequence preceded the KDEL sequence, significant amounts of the reporter protein and activity were detected in the conditioned media (see FIG. 3, lane 4). The ability of the reporter molecule to be secreted when the SEVNLDAEF (SEQ ID NO:4) (the Swedish mutation of amyloid precursor) sequence is present in the construct is consistent with the published literature. The Swedish mutation of APP has been reported to be a much better substrate, or protease cleavage site, for BACE compared to the wild type protease cleavage site.

The presented data demonstrate that 5,417,730 photons (100%) of alkaline phosphatase activity were detected in the conditioned media when secreted alkaline phosphatase was transfected into neuro-2 cells. In contrast, when the recombinant molecule had a-KDEL at the C-terminus in addition to the SEVKAMDAEF (SEQ ID NO:3) sequence, the protein was retained intracellularly (see western blot of cell extract, FIG. 4) and the amount of phosphatase activity detected in the media constituted only 1.6% of the control (no-KDEL) or 83,704 photons. Interestingly, when the SEVNLDAEF (SEQ ID NO:4) was included with the KDEL sequence, the efficiency of secretion significantly increased to 66% of control or 3,463,800 photons.

The above data derived from experiments using an alkaline phosphatase activity assay are in complete agreement with data obtained by western blot analysis using an alkaline phosphatase antibody. All three constructs were tested: secreted alkaline phosphatase (sAP), secreted alkaline phosphatase with the wild type BACE cleavage site (SEVKAMDAEF (SEQ ID NO:3) of amyloid precursor (sAP-KDELwt) as well as the molecule which contains secreted alkaline phosphatase fused to the Swedish mutation of the BACE cleavage site in APP (SEVNLDAEF (SEQ ID NO:4) and the KDEL sequence (sAP-KDELswe). All three molecules were synthesized and present within the cell (see gel for cell extracts, FIG. 4) but the molecule that lacked the KDEL sequence was secreted efficiently into the media while the sAP-KDELwt molecule was not detected in the media (FIG. 3). In support of previously published results that the Swedish mutation results in efficient cleavage by BACE, the sAP-KDELswe molecule was secreted efficiently due to cleavage by BACE.

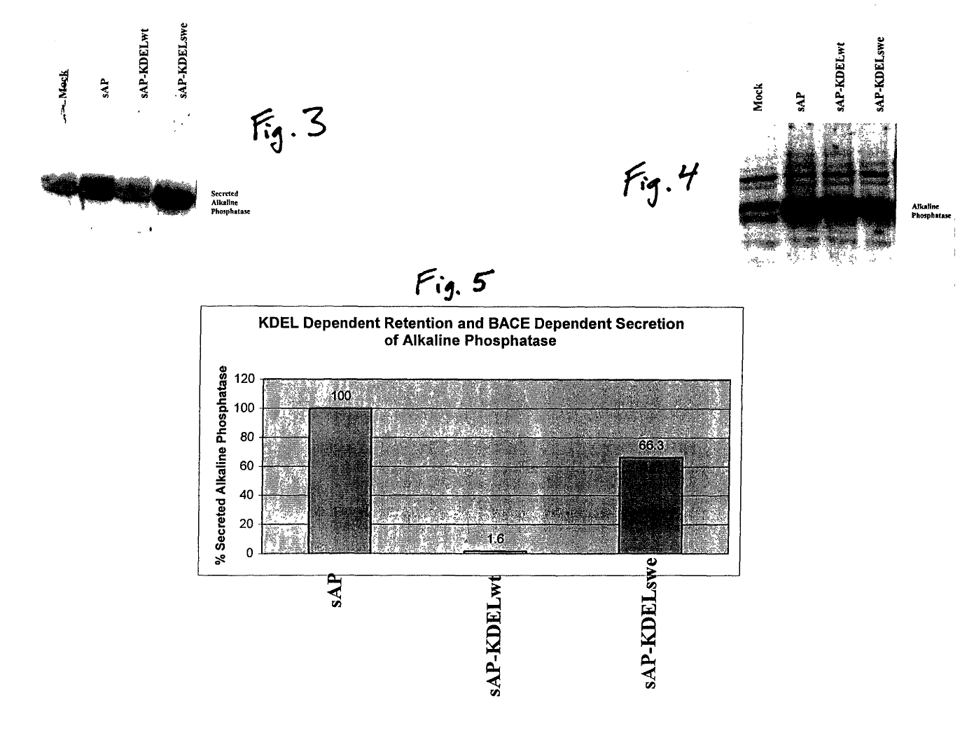

The stable cell line of the invention that expresses the sAP-KDELswe mutation is a powerful tool to screen for modulators of BACE, including inhibitors of BACE activity. An inhibitor of BACE would inhibit ER resident BACE activity, which would thus result in a decrease in the amount of BACE activity and therefore decrease the amount of alkaline phosphates secreted into the media. See FIG. 5 for a schematic summary of KDEL dependent retention and BACE dependent secretion of alkaline phosphatase.

A number of embodiments of the invention have been described. Nevertheless, it will be understood that various modifications may be made without departing from the spirit and scope of the invention. Accordingly, other embodiments are within the scope of the following claims.

What is claimed is:

1. A chimeric nucleic acid encoding a polypeptide comprising a first, a second and a third domain, wherein the first domain comprises a Golgi retention signal peptide or an endoplasmic reticulum (ER) retention signal peptide, the second domain comprises a protease cleavage site, and the third domain comprises a reporter molecule, and the protease cleavage site is between the Golgi retention signal peptide and the reporter molecule.

2. The chimeric nucleic acid of claim 1, wherein the Golgi retention signal peptide is a mammalian, a yeast or a viral Golgi retention signal peptide.

3. The chimeric nucleic acid of claim 2, wherein the mammalian Golgi retention signal peptide is a human Golgi retention signal peptide.

4. The chimeric nucleic acid of claim 1, wherein the Golgi retention signal peptide comprises a sequence motif KDEL (SEQ ID NO:1).

5. The chimeric nucleic acid of claim 1, wherein the Golgi retention signal peptide comprises a sequence motif NEFA (SEQ ID NO:2).

6. The chimeric nucleic acid of claim 1, wherein the Golgi retention signal peptide is a Golgi retention signal peptide from a Golgi resident enzyme.

7. The chimeric nucleic acid of claim 6, wherein the Golgi resident enzym e comprises a Golgi glycosyltransferase.

8. The chimeric nucleic acid of claim 7, wherein the Golgi glycosyltransferase comprises a glucosaminyltransferase I (GlcNAcTI), a beta 1,4-galactosyltransferase (GalT) or an alpha 2,6-sialytransferase (ST).

9. The chimeric nucleic acid of claim 1, wherein the second domain comprises a sequence encoding two protease cleavage sites.

10. The chimeric nucleic acid of claim 1, wherein the first domain coding sequence is upstream of the second domain coding sequence and the third domain coding sequence.

11. The chimeric nucleic acid of claim 1, wherein the third domain coding sequence is upstream of the second domain coding sequence and the first domain coding sequence.

12. The chimeric nucleic acid of claim 1, wherein the protease cleavage site comprises a secretase cleavage site.

13. The chimeric nucleic acid of claim 12, wherein the secretase cleavage site comprises a beta-secretase cleavage site.

14. The chimeric nucleic acid of claim 13, wherein the beta-secretase cleavage site comprises a sequence SEVKMDAEF (SEQ ID NO:3).

15. The chimeric nucleic acid of claim 13, wherein the beta-secretase cleavage site comprises a sequence SEVNLDAEF (SEQ ID NO:4).

16. The chimeric nucleic acid of claim 12, wherein the secretase cleavage site comprises a gamma-secretase cleavage site.

17. The chimeric nucleic acid of claim 1, wherein the reporter molecule comprises an enzyme.

18. The chimeric nucleic acid of claim 17, wherein the enzyme comprises an alkaline phosphatase.

19. The chimeric nucleic acid of claim 1, wherein the reporter molecule comprises a fluorophore.

20. The chimeric nucleic acid of claim 19, wherein the fluorophore comprises a green fluorescent protein (GFP).

21. The chimeric nucleic acid of claim 1, wherein the reporter molecule comprises a bioluminescent or a chemiluminescent polypeptide.

22. The chimeric nucleic acid of claim 21, wherein the chemiluminescent polypeptide comprises a luciferase.

23. The chimeric nucleic acid of claim 21, wherein the bioluminescent or chemiluminescent polypeptide comprises an aequorin, an obelin, a mnemiopsin or a berovin.

24. The chimeric nucleic acid of claim 1 further comprising a promoter, wherein the chimeric polypeptide-encoding nucleic acid is operably linked to a promoter.

25. The chimeric nucleic acid of claim 24, wherein the promoter is a constitutive promoter.

26. The chimeric nucleic acid of claim 24, wherein the promoter is an inducible promoter.

27. An expression cassette comprising a chimeric nucleic acid encoding a polypeptide comprising a first, a second and a third domain, wherein the first domain comprises a Golgi retention signal peptide or an endoplasmic reticulum (ER) retention signal peptide, the second domain comprises a protease cleavage site, and the third domain comprises a reporter molecule, and the protease cleavage site is between the Golgi retention signal peptide and the reporter molecule.

28. An expression vector comprising a chimeric nucleic acid encoding a polypeptide comprising a first, a second and a third domain, wherein the first domain comprises a Golgi retention signal peptide or an endoplasmic reticulum (ER) retention signal peptide, the second domain comprises a protease cleavage site, and the third domain comprises a reporter molecule, and the protease cleavage site is between the Golgi retention signal peptide and the reporter molecule.

29. A transformed host cell comprising a nucleic acid encoding a chimeric polypeptide comprising a first, a second and a third domain, wherein the first domain comprises a Golgi retention signal peptide or an endoplasmic reticulum (ER) retention signal peptide, the second domain comprises a protease cleavage site, and the third domain comprises a reporter molecule, and the protease cleavage site is between the Golgi retention signal peptide and the reporter molecule.

30. The transformed host cell of claim 29, wherein the cell is a bacterial cell, a mammalian cell, a yeast cell, an insect cell or a plant cell.

31. A non-human transgenic animal comprising a nucleic acid encoding a chimeric polypeptide comprising a first, a second and a third domain, wherein the first domain comprises a Golgi retention signal peptide or an endoplasmic reticulum (ER) retention signal peptide, the second domain comprises a protease cleavage site, and the third domain comprises a reporter molecule, and the protease cleavage site is between the Golgi retention signal peptide and the reporter molecule.

32. A non-human transgenic animal that expresses a chimeric polypeptide comprising a first, a second and a third domain, wherein the first domain comprises a Golgi retention signal peptide or an endoplasmic reticulum (ER) retention signal peptide, the second domain comprises a protease cleavage site, and the third domain comprises a reporter molecule, and the protease cleavage site is between the Golgi retention signal peptide and the reporter molecule

33. The non-human transgenic animal of claim 31, wherein the animal is a mouse or a rat.

34. The non-human transgenic animal of claim 32, wherein the animal is a mouse or a rat.

35. A chimeric polypeptide comprising a first, a second and a third domain, wherein the first domain comprises a Golgi retention signal peptide or an endoplasmic reticulum (ER) retention signal peptide, the second domain comprises a protease cleavage site, and the third domain comprises a reporter molecule, and the protease cleavage site is between the Golgi retention signal peptide and the reporter molecule.

36. A chimeric polypeptide comprising a first, a second and a third domain, wherein the first domain comprises a Golgi retention signal peptide or an endoplasmic reticulum (ER) retention signal peptide, the second domain comprises a beta-secretase protease cleavage site, and the third domain comprises an alkaline phosphatase or a green fluorescent protein (GFP) reporter molecule, and the protease cleavage site is between the Golgi retention signal peptide and the reporter molecule.

37. A kit comprising a chimeric nucleic acid as set forth in claim 1 or a polypeptide as set forth in claim 35 and instructions for use.

38. The kit of claim 37, further comprising a substrate for a bioluminescent or chemiluminescent polypeptide or the alkaline phosphatase.

39. The kit of claim 37, wherein the instructions are for measuring protease activity in vivo.

40. A method for detecting a protease activity comprising
(a) expressing a chimeric nucleic acid as set forth in claim 1 in a cell, or placing a chimeric polypeptide as set forth in claim 35 in a cell; and
(b) detecting the amount of reporter molecule secreted by the cell, thereby detecting a protease activity.

41. A method for identifying a modulator of a protease activity comprising
(a) providing a test compound;
(c) expressing a chimeric nucleic acid as set forth in claim 1 in a cell, or placing a chimeric polypeptide as set forth in claim 35 in a cell;
(c) detecting the amount of reporter molecule secreted by the cell;
(d) exposing the cell to the test compound and detecting the amount of reporter molecule secreted by the cell;
(e) comparing the amount of reporter molecule secreted by the cell before exposure to the test compound to the amount of reporter molecule secreted by the cell after exposure to the test compound, wherein a difference in amounts identifies the test compound as a modulator of protease activity.

42. The method of claim 41, wherein the cell is a bacterial cell, a mammalian cell, a yeast cell, an insect cell or a plant cell.

43. The method of claim 41, wherein the cell is in a tissue culture media, and detecting the amount of reporter molecule secreted by the cell comprises measuring the amount of report molecule in the tissue culture media.

44. The method of claim 41, wherein the cell is a transgenic cell comprising and expressing a chimeric nucleic acid as set forth in claim 1.

45. The method of claim 41, wherein a decrease in the amount of reporter molecule secreted by the cell after exposure to the test compound identifies an inhibitor of the protease.

46. The method of claim 41, wherein an increase in the amount of reporter molecule secreted by the cell after exposure to the test compound identifies an activator of the protease.

47. The method of claim 41, wherein the protease cleavage site comprises a secretase cleavage site.

48. The method of claim 47, wherein the secretase cleavage site comprises a beta-secretase cleavage site.

49. The method of claim 48, wherein the beta-secretase cleavage site comprises a sequence SEVKMDAEF (SEQ ID NO:3).

50. The method of claim 47, wherein the secretase cleavage site comprises a gamma-secretase cleavage site.

51. A method for detecting a protease activity in an intact non-human animal comprising
(a) expressing a chimeric nucleic acid as set forth in claim 1 in a cell in the animal, or placing a chimeric polypeptide as set forth in claim 35 in a cell in the animal; and
(b) detecting the amount of reporter molecule secreted by the cell, thereby detecting a protease activity in an intact non-human animal.

52. The method of claim 51, wherein expressing the chimeric nucleic acid in a cell comprises administering to the non-human animal an expression vector or a recombinant virus comprising the chimeric nucleic acid.

53. The method of claim 51, wherein the non-human animal comprises a transgenic non-human animal comprising the chimeric nucleic acid.

54. A method for identifying a modulator of a protease activity in an intact non-human animal comprising
(a) providing a test compound;
(c) expressing a chimeric nucleic acid as set forth in claim 1 in a cell in the animal, or placing a chimeric polypeptide as set forth in claim 35 in a cell in the animal;
(c) detecting the amount of reporter molecule secreted by the cell;
(d) exposing the animal to the test compound and detecting the amount of reporter molecule secreted by the cell;
(e) comparing the amount of reporter molecule secreted by the cell before exposure to the test compound to the amount of reporter molecule secreted by the cell after exposure to the test compound, thereby identifying a modulator of a protease activity in an intact non-human animal.

55. The method of claim 54, wherein detecting the amount of reporter molecule secreted by the cell comprises taking a fluid sample from the animal and measuring the amount of reporter molecule in the fluid sample.

56. The method of claim 55, wherein the fluid comprises taking a blood sample, a cerebral spinal fluid sample, a saliva sample or a urine sample.

57. The method of claim 54, wherein detecting the amount of reporter molecule secreted by the cell comprises taking a tissue sample from the animal and measuring the amount of reporter molecule in the tissue sample.

58. The method of claim 57, wherein the tissue sample comprises a biopsy sample.

59. The method of claim 54, wherein the reporter molecule comprises a molecule that can be directly or by enzymatic reaction with a reagent generate a molecule that can be imaged by computer assisted tomography (CAT), magnetic resonance spectroscopy (MRS), magnetic resonance imaging (MRI), positron emission tomography (PET), single-photon emission computed tomography (SPECT), bioluminescence image (BLI) or equivalent.

60. The method of claim 59, wherein the reporter molecule comprises a bioluminescent or a chemiluminescent polypeptide.

61. The method of claim 60, wherein the chemiluminescent polypeptide comprises a luciferase.

62. The method of claim 60, wherein the bioluminescent or chemiluminescent polypeptide comprises an aequorin, an obelin, a mnemiopsin or a berovin.

63. The method of claim 54, wherein the reporter molecule comprises a green fluorescent protein.

64. The method of claim 54, wherein the reporter molecule comprises an alkaline phosphatase.
